(19) United States
(12) Patent Application Publication
(10) Pub. No.: US 2026/0364357 A1
(43) Pub. Date: Nov. 19, 2026

(54) INTRAVASCULAR CATHETER FOR ADAPTIVE CONTROL OF CARDIAC PACING BASED ON RESPIRATORY AND HEMODYNAMIC INDICES

(71) Applicant: Levron Cardiovascular Ltd., Yokneam Illit (IL)

(72) Inventor: Amir LANDESBERG, Haifa (IL)

(73) Assignee: Levron Cardiovascular Ltd., Yokneam Illit (IL)

(21) Appl. No.: 19/135,421

(22) PCT Filed: Dec. 4, 2023
(86) PCT No.: PCT/IB2023/062209

§ 371 Date: Jun. 4, 2025

Related U.S. Application Data
(60) Provisional application No. 63/385,975, filed on Dec. 4, 2022.

(51) Int. Cl. A61N 1/365

(52) U.S. Cl. CPC A61N 1/36585

(57) ABSTRACT

An intravascular catheter monitors respiratory and hemodynamic indices and simultaneously delivers cardiac stimulations enables to perform modulation of a cardiac rhythm relative to the pleural pressure, for adaptive treatment of heart failure, low cardiac output or lung congestion. The catheter enables measuring the instantaneous pleural pressure, based on measurements of the intravascular volume and pressure of an intrathoracic large elastic vessel.

### FIELD OF THE INVENTION

The invention relates to a novel method for sensing respiratory and hemodynamic conditions and pacing the heart, utilizing a single catheter or several catheters, for treatment of heart-failure, low cardiac output or pulmonary congestion. The catheter enables measuring the instantaneous pleural pressure, based on measurements of the intravascular volume and pressure of an intrathoracic large elastic vessel. The invention may be used for treatment of cardiac decompensation, amelioration of dyspnea and prevention of deterioration to pulmonary congestion and pulmonary edema. It applies to patients with acute or chronic heart failure from any etiology.

### BACKGROUND OF THE INVENTION

Heart failure is a leading pandemic that is associated with poor quality of life, high morbidity and mortality. There are various treatments for heart failure: various drugs, resynchronization of the myocardial contraction by pacing the heart at different sites (CRT, cardiac resynchronization therapy), cardiac contractility modulation, neurohumoral stimulation, mechanical assist devices, attempts to use stem-cell therapy for myocardial regeneration and utilization of various materials for tissue rejuvenation. Despite the significant advances in all the suggested technologies and therapies the mortality and morbidity are still high and quality of life is very poor. Moreover, the prevalence of heart failure is expected to grow in the near future with the aging of the population.

The main problem of all known solutions for the treatment of heart failure is that their effectiveness is very limited. The current solutions have only limited success in altering the course of the disease, although they may ameliorate of rate of heart failure progression. Few thousands of assist devices are implanted every year in patients with end-stage heart-failure. Although this technology significantly prolongs their survival, these patients are only a small fraction of the population that suffer from severe heart-failure, few thousands out of a population of more than 1.5 million patients with stage III-IV heart failure, in the USA alone. Moreover, the assist device technology is associated with high rate of complications as gastrointestinal bleeding, stroke, and right-heart failure. In addition, it is a very expensive technology.

Cardiac Resynchronization Therapy (CRT) is another advanced technology, but it is effective in only a small fraction of the patients with severe systolic heart failure (patients with ejection fraction below 35% and markedly prolonged QRS). Interestingly, more than half of the heart failure patients suffer from diastolic heart-failure, a problem in the filling of the left ventricle. The mechanisms underlying this type of heart failure are not well-understood and there is no effective remedy to this type of heart failure. This group of diastolic heart failure (or heart failure with preserved ejection fraction) is steadily growing with the aging of the population. Therefore, there is a crucial unmet need to develop novel technologies for the treatment of heart failure.

Three main paradigms have been suggested to explain the development of heart failure: (1) cardio-renal and volume overload, (2) cardio-circulation and coupling the cardiac function with the peripheral impedance, (3) neuro-humoral and the activation of the sympathetic system. The current available drug therapies and the various technologies relate to these three paradigms.

### SUMMARY

The present invention presents novel methods and devices for treatment of heart-failure and pulmonary congestion and also to prevention of atrial fibrillation; without limitation, the invention may be used for treatment of cardiac decompensation, amelioration of dyspnea and prevention of deterioration to pulmonary congestion and pulmonary edema.

The most cardinal complaint of severe heart failure and the main cause for rehospitalization is severe dyspnea. The inventor has found that the respiratory effort and the associated sensation of dyspnea are not only hallmarks of cardiac decompensation but the respiratory effort plays a pivotal role in the ‘vicious cardiopulmonary cycle’ that can lead to progressive deterioration.

The novel small implantable device provides continuous adaptive treatment based on real time monitoring and diagnosis. The device monitors the respiratory effort. It utilizes the opposite effects of the respiratory effort on right and left ventricle outflows. Inspiration or negative intrathoracic pressure increases the right-side outflow into the lung while decreasing the drainage to the left ventricle and cardiac output. The opposite occurs during expiration. Development of cardiac decompensation aggravates these effects of the respiratory effort on the right and left ventricles output, leading to the development of “vicious cardiopulmonary cycle”; an increase in the respiratory effort accelerates the development of lung congestion that further decreases lung compliance, while the latter increases the respiratory effort, and the cycle repeats itself. The novel device modulates the cardiac rhythm and changes the distribution of the heart beats at the various pleural pressures. By pacing the heart when the pleural pressure is close or above zero, it increases the probability of heart beating at end expiration and decreases the probability of heart beating at deep negative pleural pressure. The changes in the cardiac rhythm increase the number of heart beats with high left-ventricle stroke volume and low right ventricle stroke-volume. Consequently, it breaks the vicious cardiopulmonary cycle and shifts blood and fluids from the lung to the periphery and alleviates lung congestion and dyspnea. It also alleviates the workload of the heart and improves the cardiac output and cardiopulmonary endurance. The feasibility was validated in healthy sheep and sheep with acute HF induced by acute infarction. On average the device shifts 0.5 ml of blood from the lung per one breath cycle. Pacing only 2% of the time, 400 breath-cycles out of the daily 20,000 breath cycles, can shift 200 ml per day. The pacing is adaptive to the patient's needs. As congestion develops slowly over few weeks, the device can provide early detection and prevent development of congestion and dyspnea.

The novel treatment is a breakthrough in the management of heart failure for the following main reasons:
1. It is based on a novel paradigm for understanding the deterioration of heart failure.
2. It counteracts the normal physiological control of cardiac pacing.
3. It is independent of the various etiologies of heart failure, and can treat all of them.
4. It provides assessment of the severity of cardiac decompensation and immediately provides a treatment that is proportional to the severity of the decompensation.
5. It can detect early deterioration and provide treatment before the patient may become symptomatic-provides personalized medicine with early detection and prevention.

Advantages of the invention include, without limitation:
1. It applies to the huge market of heart failure, and can be used in all the patients with stage 3 and 4 heart failure.
2. It applies to all forms of heart failure, independently of the etiology, whether it is heart failure with reduced ejection fraction or preserved ejection fraction.
3. It provides immediate treatment to the detected development of dyspnea or an increase in the intrapulmonary blood pressure (hemodynamic congestion).
4. It can detect the slow progression of the disease and treat it before the patients become symptomatic. Thus, it provides prevention and can decrease the rate of hospitalization.
5. It represents novel autonomic control (diagnosis and treatment) of the human autonomic cardiac system. It enables tight continuous surveillance of the heart-failure patients, with tight monitoring of the changes in the hemodynamic congestion and the effectiveness of the treatment.
6. It is simple to implement. It is based on integration of pacing technology with sensing the respiratory effort and a novel algorithm.
7. It requires low power because it utilizes the power of the “respiratory pump” and the cardiac contraction to push blood out of the lung and to alleviate the hemodynamic congestion.
8. The expected adverse effects are low. The adverse effects may relate to the presence of a pacing electrode within the heart and to the modulation of the heart rate, and both have well-known low rate of adverse effects. Modulation of the heart rate is not expected to have any adverse effect, since patients with atrial fibrillation with rate control has the same prognosis as patients with tight rhythm control.

The present invention describes a novel intravascular catheter that can be used for both monitoring the respiratory and hemodynamic conditions and pacing the heart. The catheter is connected to a device that can modulate the cardiac rhythm and synchronize it to the respiratory phases and the pleural pressure. The catheter delivers the required inputs to the device and transmits the device pacing output to the heart. It enables to measure the instantaneous pleural pressure, based on measurements of the intravascular volume and pressure of an intrathoracic large elastic vessel. The catheter is used for treatment of heart failure, low cardiac output and pulmonary congestion and also for prevention of atrial fibrillation; without limitation, the invention may be used for treatment of cardiac decompensation, amelioration of dyspnea and prevention of deterioration to pulmonary congestion and pulmonary edema.

The present invention includes methods and systems for sensing, diagnosing, and/or treating respiratory and hemodynamic conditions and for pacing the heart using a single catheter that includes one or more sensors and one or more pacing electrodes.

The pacing electrodes may be at the tip of the catheter which is implanted in one of the heart chambers (atria or ventricle, right or left). The sensors along the catheter may be positioned within the venous circulation or the cardiac chamber.

The sensors along the catheter may provide two type of information: sensing the cardiac and respiratory rhythm for real time control of the timing of the appropriate pacing, and sensing long-term hemodynamic and respiratory indices for assessing the severity of cardiac decompensation and respiratory effort.

Sensing the cardiac rhythm and indices of cardiac decompensation may be done by sensing the cardiac autonomous electrical activity by the electrodes at the distal tip of the catheter, for (a) appropriate timing of pacing in cadence with cardiac autonomous pacing, and (b) monitoring the cardiac electrophysiological conditions by detecting arrhythmias and analyzing the changes in heart-rate variability as indices of the autonomic system activity and severity of cardiac decompensation.

Assessing the severity of cardiac decompensation may be done by pressure transducer along the catheter, for sensing the severity of hemodynamic congestion and the increase in the central venous pressure or cardiac camber pressure.

Sensing the respiratory rhythm and indices of cardiac decompensation may be done by a pressure transducer positioned along the catheter, for (a) appropriate timing of pacing in cadence with respiratory cycle, by detecting the transient pressure changes due to inspiration and expiration, (b) monitoring the respiratory rate and the pattern of respiration as indices of the severity of cardiac decompensation.

Sensing indices of cardiac decompensation may be done by a pressure transducer positioned along the catheter that provides the modulation of the intravascular pressure due to respiration, for the assessment of the peak-to-peak changes in the intrathoracic pressure and the patient respiratory effort.

Sensing indices of cardiac decompensation may be done by measuring the changes in the vessel diameter or the changes in the volume in a segment of the vena-cava or within the cardiac chamber, as an index for the severity of blood congestion. The changes in the volume are monitored by measuring the changes in the electrical impedance. An increase in the vessel diameter decreases the impedance. For a well-defined segment, which is defined by the distance between the two sensing electrodes, the impedance is a hyperbolic function of the volume.

The measurement of changes in the vascular impedance may be done with four electrodes along the catheter (and four wires), wherein the two distal electrodes are used for injecting alternate currents, and the two central electrodes are used for sensing the voltage. It can be implemented also by only two electrode (two wires), that imposed alternating voltages and measure the current. The current is directly proportional to volume within the vessel.

Sensing indices of cardiac decompensation may include the calculation of the respiratory-induced changes in the pleural pressure, an index of the respiratory effort and severity of cardiac decompensation, based on the measured intravascular pressure and the simultaneous changes in the intravascular blood volume.

Sensing indices of cardiac decompensation may be done by measuring the changes in lung congestion by measuring the impedance between electrodes along the catheter, deep within the chest cavity and electrode on the pacemaker/controller.

Monitoring the respiration may include monitoring the changes in the respiratory pattern as the changes in the ration between the inspiratory and expiratory intervals.

The pressure sensors may be, without limitation, embedded solid state strain-gauge (with two electrical wires) or half of a Wheatstone bridge (three wires), where only one gauge is exposed to the pressure and the other is used for a reference and temperature adjustments, or alike.

Each of the pressure sensors may be a flexible membrane with a thin lumen that transmits the pressure to a pressure transducer at the proximal end of catheter, within the connector to the implanted pacemaker/controller, or even within the pacemaker.

The catheter includes a thin lumen for a wire guide, needed for the position and implantation of the catheter to the wall of cardiac chamber.

The distal end of the catheter includes a controlled spring, which enables safe and secured implantation into the myocardium. This spring can also serve as the pacing electrode.

Some non-limiting features of the invention include:
1. A single catheter (FIG. 3) for both sensing (10, 11, 12) and pacing (8, 9).
2. Sensing signals include a combination of the following signals:
a. The ECG (8 in the figures)—monitoring changes in heart rate, heart rhythm and ECG axis
b. Pleural pressure (P_{PL})
c. Blood pressure within intrathoracic blood vessel or within the heart (12 in the figures)
d. Volume changes in intrathoracic vessel, heart chamber, of vena-cava (10, 11 in 30 the figures).
e. Measurement of changes in the pulmonary impedance (13 in the FIGS.
3. The ECG is essential for the precise triggering of cardiac pacing, in the appropriate time after the last cardiac QRS signal.
4. The peak-to-peak amplitude of the pleural pressure is used as a surrogate to the respiratory effort.
5. The pleural pressure, intravascular volume, and intravascular pressure, pulmonary impedance and changes in the cardiac rhythm are used in combination or separately to define the respiratory phases and the severity of the respiratory effort.
6. The pleural pressure (P_{PL}) is assessed from the measurements of the intravascular volume (V_{V}) and pressure (P_{V}), where C_{V} is the vessel compliance, and it is constant.
7. The vessel compliance (C_{V}) can be estimated noninvasively, by asking the patient to stop breathing at least two times, and to generate different mouth pressure each time. From two simultaneous measurements of intravascular volume (V_{V1}, V_{V2}), intravascular pressure (P_{V1},P_{V2}) and the pressure at the mount (P_{M1},P_{M2}) it is possible to calculate the vessel compliance:
8. The thoracic impedance can be measured from the each of the catheter electrodes and the device (13 in the figures).
9. The catheter can be implanted in each of the heart chambers (4, 5, 6, or 7). The preferred site is the right atrium (4) if the patient has normal sinus rhythm. If the patient has atrial fibrillation the preferred site is the right ventricle (5).
10. Several catheters can be used, some dedicated for the sensing and some dedicated for pacing. Sensing can be made simultaneously in the vena cava and some of the heart chamber (as the right atrium and right ventricle). Pacing can be done in both the 20 atrium and the ventricle in case of atrioventricular block.

It is noted that the term “catheter” encompasses any slender element suitable for mounting thereon or therein a pressure sensor and electrode.

### BRIEF DESCRIPTION OF THE DRAWINGS 25

The present invention will be understood and appreciated more fully from the following detailed description taken in conjunction with the drawings in which:

FIG. 1 is a simplified illustration of “respiratory sinus arrhythmia”, which is a normal physiological increase in the heart rate during inspiration and decrease in the heart rate during expiration (depicted by the arrows). The cardiopulmonary reverse cycling therapy of the invention counteracts with the normal physiology and increases the heart rate only when the intrathoracic pressure is close to zero, at end expiration and the beginning of inspiration. During normal breathing there is a progressive increase in the heart rate and inflow into the lung during inspiration that is compensated by an increase in the outflow during expiration. In contrast the proposed cardiopulmonary reverse cycling therapy causes a net decrease in hemodynamic and lung congestions, by increasing the absolute number of cardiac contractions during time intervals with close to zero intrathoracic pressure over time interval with deep negative intrathoracic pressure.

FIG. 2 illustrates experimental validation, in sheep, of the effects of breathing on the right and left ventricle stroke-volumes. The changes in the pleural pressure have overt opposite effects on two ventricles. The decrease in the pleural pressure during inspiration is associated with an increase in the right-ventricle stroke-volume and simultaneous decrease in the left-ventricle stroke-volume. The maxima right ventricle output and the minimal left ventricle stroke volumes are reached at the deepest negative pleural pressure, at end-inspiration. Expiration yields the opposite, a decrease in the right-ventricle output and an increase in the left-ventricle output. Thus, pacing at mid and late expiratory phase promotes draining blood out of the lung through the left ventricle and decreases blood inflow into the lung through the right ventricle.

FIG. 3 depicts an example of the inventive synchronization of the cardiac pacing to the changes in the intrathoracic pressure and swings in the respiratory wave, based on data from heart failure patient. The upper bars denote the imposed pacing (red bars) over the native regular sinus pacing (blue bars) on top of the recorded ECG. The lower trace presents the measured changes in the intrathoracic pressure. Note the patient suffers from severe dyspnea with respiratory effort (peak to peak amplitude) of 15 mmHg, about 5-fold the normal respiratory effort. The novel algorithm sets a threshold (at −5 mmHg in this example) and segments the respiratory cycles into two types of time intervals: (1) Time intervals with deep negative intrathoracic pressure (red), at end inspiration and early expiration. (2) Time intervals with intrathoracic pressure close to zero (green). The system adds excitations when the intrathoracic pressure is close to zero (green), to provide cardiopulmonary reverse cycling and to reduce the hemodynamic and lung congestions.

FIGS. 4 and 4A illustrate different positions of the catheter. The device (1) can be implanted under the pectoralis muscle, in the left upper chest, as a regular pacemaker. The catheter (3) in inserted through the subclavian vein and upper vena-cava (2) into the right-atrium (4), in the described embodiment. Pacing in the right atrium is the preferred mode of pacing for patient with normal sinus rhythm. However, the catheter can be inserted into other chambers of the heart (5—right ventricle, 6—left atrium, 7—left ventricle) and also several similar catheters can be used. The catheter is implanted in the right ventricle when the patient suffers from atrial fibrillation. Two catheters may be used, one in the right atrium and a second in the right ventricle when the patient suffers from atrioventricular conduction abnormalities.

FIGS. 5, 5A and 5B illustrate more detailed structure of the catheter (3). The catheter can deliver cardiac stimulation by the electrode of electrodes at distal end of the catheter (8, 9). The distal end (9) may include a sharp spring, which is inserted into the myocardium during the implantation, and stabilizes the position of the catheter. Cardiac pacing can be provided by a single electrode (between the distal end a second reference electrode on the device surface), or by bipolar electrodes (8 and 9) at the distal end of the catheter. The spring can serve as the electrode.

The sensors are positioned along the catheter, to enable measurements of the intravascular pressure, intravascular volume and the impedance between the electrodes and the device. For the measurement of the intravascular pressure and volume in the vena cave the sensors (10, 11, and 12) are placed at least 8-10 cm from the distal end. The intravascular volume is monitored by measuring the changes in the impedance (14—the electrical potential waves) between two close electrodes (10, 11), within the vena cava. The intravascular pressure in measured by a miniature pressure transducer that is placed between the impedance electrodes, to ensure that the pressure and volume are measured from the same vascular segment. The pleural pressure is calculated based on these simultaneous measurements of the intravascular pressure and volume. Changes in the pulmonary congestion can be sensed by measuring the changes in the pulmonary impedance between any of the catheter electrodes and the device (13).

### DETAILED DESCRIPTION

The invention enables simple implementation of a novel method for providing adaptive treatment for heart failure, low cardiac output and pulmonary edema, utilizing a single catheter for both sensing and pacing. The novel therapy is called herein the “cardiopulmonary reverse cycle” or “breaking the vicious cardiopulmonary cycle” and it is doing it by changing the cardiac rhythm and slightly synchronizing the cardiac contractions to the different breathing phases.

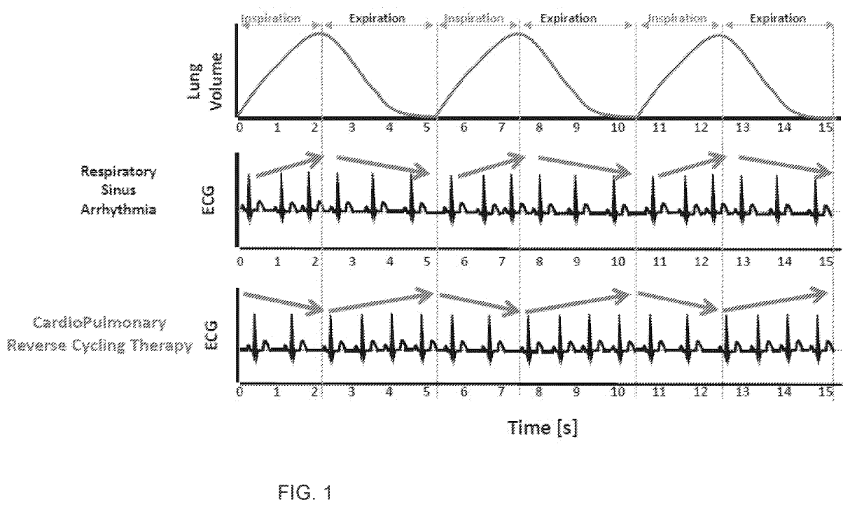

The normal physiological control of the heart rate aims to increase the cardiac output during exercises, and it is done efficiently in healthy subjects. An increase in the respiratory work by the “respiratory pump” (the diaphragm and all the respiratory and accessory muscles), decreases the intrathoracic pressure during inspiration and facilitates venous return to the right atrium. The cardiac output of the left ventricle is equal to the venous return, at steady state, and is limited by the venous return. Under normal physiological conditions, an increase in the venous return and the ensuing dilatation of the right atrium increases both the heart rate of the sinus node and increases the stroke volume based on the Fran-Starling Law of the heart. There is an increase in the heart rate especially during inspiration, since the venous return increases during inspiration. This phenomenon denoted is denoted as the ‘respiratory sinus arrhythmia’ and is depicted in FIG. 1. The increase in the cardiac output during exercise is due to an increase in the venous return to the heart, increase in the heart rate and the stroke volume of both ventricles. Thus, the “respiratory pump” has significant effects on cardiac function.

However, while the respiratory effort of the “respiratory pump” increases the cardiac output under normal physiological condition, an increase in the respiratory effort has several severe detrimental effects on the pulmonary circulation and the cardiac workloads, especially in the setting of heart failure. The increase in the respiratory effort increases the: (1) intrapulmonary capillary pressure (PCWP), (2) pulmonary vascular resistance (PVR), pulmonary artery pressure (PAP) and right-ventricle afterload, (3) lung congestion by shifting blood into the lung, (4) LV afterload, and (5) metabolic demand due to the increase in the work of the respiratory muscles. All these mechanisms are detrimental in the setting of heart failure. These five adverse effects of an increased respiratory effort lead to accelerated decompensation in the presence of heart or severe lung diseases.

The main strategy behind the present innovation is to utilize the “respiratory pump” and cardiac contractions in order to pump blood out of the lung and to alleviate the hemodynamic congestion. It is done by modulating cardiac pacing according to the changes in the pleural pressure, but counterintuitively, it is works opposite to the normal physiology and increases the heart rate during end expiration and early inspiration, as depicted in FIG. 1 (Reversed Cardiopulmonary Cycling Therapy). During late expiration and early inspiration, when the “respiratory pump” decreases the right-ventricle preload, decreases the left ventricle afterload and increases the left-ventricle preload, the device increases the pacing rate to facilitate the removal of blood from the lung. It is also desired to decrease the heart rate during end inspiration and early expiration, when the pleural pressure is negative and the “respiratory pump” increases the right-ventricle preload and the left-ventricle afterload.

The pressures in the pulmonary circulation (hemodynamic congestion) and the amount of blood and fluids with the lung (lung congestion) are mainly determined by the inflow of blood into the lung through the right ventricle and the outflow of blood out from the lung back into the peripheral circulation through the left ventricle. However, these inflow and outflow, through the right and left ventricle, are modulated by the intrathoracic pressure. In the presence of a deep negative intrathoracic pressure the inflow into the lung is larger than the outflow from the lung. The opposite occurs in the presence of close to zero and positive intrathoracic pressure. Therefore, the inflow and outflow are modulated by the respiratory pump.

The device utilizes the works that are generated by the respiratory system (the “respiratory pump”) and cardiac contractions in order to remove fluids from the lung. It utilizes the effects of the pleural pressure generated by the “respiratory pump”, to “pump” out of the lung and to reduce the workloads on the left ventricle. It is done by pacing the heart and increasing the number of heart beats when the intrathoracic pressure is close to zero relative to the number during deep negative intrathoracic pressure. Pacing the heart when the intrapleural pressure is close to or above zero also decreases the probability of cardiac contraction when the surrounding pleural pressure is negative, due to decrease in the sinus node rate, as was observed in our pre-clinical studies. The shift of cardiac contraction probability to higher surrounding pleural pressure facilitates left-ventricle outflow and blood drainage from the lung and simultaneously decreases the right-ventricle outflow and blood inflow into the lung. The net effect is decreases in the lung blood volume, intrapulmonary vascular pressure, pulmonary vascular resistance and pulmonary congestion. It decreases the cardiac workload by decreasing the (1) LV afterload, when the LV contract from higher surrounding pressure, (2) LV preload, by gradual decreasing lung congestion and the pulmonary venous pressure, and (3) RV afterload, by decreasing pulmonary congestion and pulmonary vascular resistance.

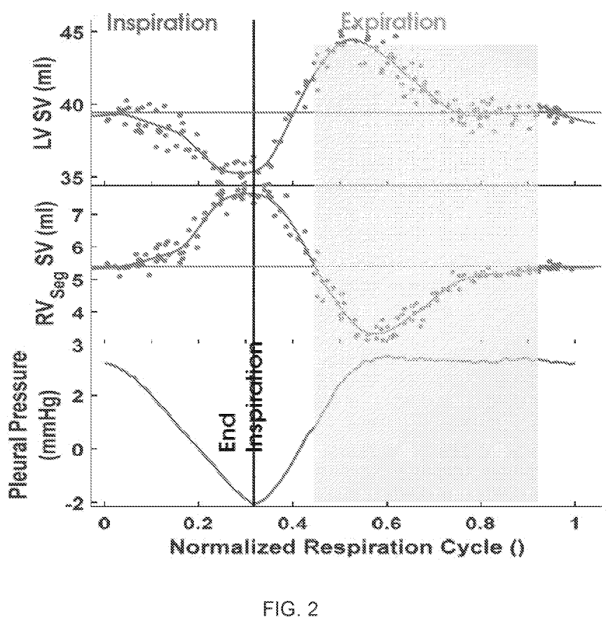

The mean cardiac stroke volume of an adult is about 70 ml. Our preclinical studies in sheep reveled huge effects of the pleural pressure on the stroke-volumes of the two ventricles, as depicted in FIG. 2. The LV output decreases by about 10% at end-inspiration and increases by 10% above the mean at mid-expiration (FIG. 2). Based on our recent preclinical data, pacing when the pleural pressure is high provide a net shift of 0.5 ml of blood out of the lung in each breath cycle. Thus, to shift 200 ml of blood out of the lung one has to pace 400 breath cycles. However, in a single day one has on average about 20,000 breath cycles and 100,000 heart beats. Thus, only a modest pacing of 2% of the breath cycles, once every 50 breath cycles or approximately every 3 minutes is needed. It is well-established the cardiac decompensation of chronic heart failure patient develops and progressed slowly over a period of 2 to 3 weeks, before there is a need for more aggressive treatment or hospitalization. Thus, although the suggested method causes a small shift of fluid from the lung back to the periphery, it can work slowly and persistently over a long-time interval of days, and thus can prevent the gradual development of lung congestion.

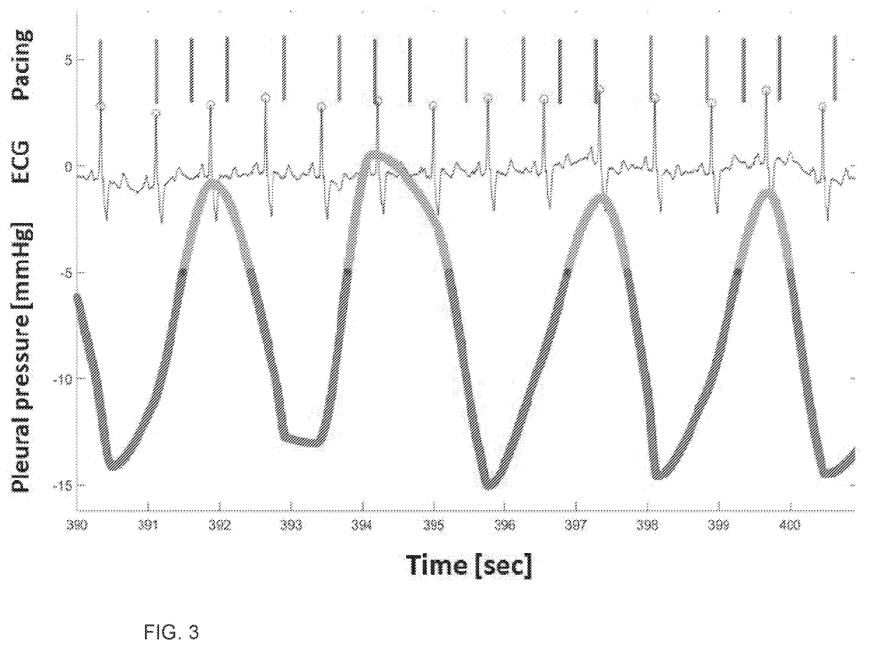

No prior art has related to the suggested crucial role of the respiratory effort in the development of cardiac decompensation. Some patents have suggested the opposite, to improve the cardiac output by pacing the heart during inspiration. The invention is based on a counterintuitive mode of pacing the heart, which is opposite to the physiological autonomic modulation of the cardiac pacing by the respiration, which is denoted as “respiratory sinus arrhythmia”. While in the physiological respiratory sinus arrhythmia the heart rate increases during inspiration, when the intrathoracic pressure drop down to the deepest negative pressure, the mode suggested here performs the opposite mode of pacing and increases the heart rate when the intrathoracic pressure is close to zero, as depicted in FIG. 3. Pacing during the inspiratory phase as suggested by other patents U.S. Pat. Nos. 8,509,902, 8,483,833) may only accentuates the vicious cycle and lung congestion, since pacing at low negative intrathoracic pressure increases the right-ventricle output, but increases the failing left-ventricle afterload and decreases the left-ventricle filling. This this mode can ultimately lead to progressive lung congestion and cardiac decompensation.

No prior art relates to intravascular catheter that is used for simultaneous monitoring of respiratory and hemodynamic indices and pacing the heat. The utilization of a single catheter simplifies the implantation procedure and significantly reduces the immediate and long-term complications.

US Patent Application 2021/0068667 (“Quantification of the respiratory effort from hemodynamic measurements”) presents a method for assessing the respiratory effort form continuous measurement of the intravascular pressure. However, that method suffers from low temporal resolution, since it is based on decomposition of the intravascular pressure signal to cardiac and respiratory signals. The current innovation has two major advances: (1) simultaneous sensing and pacing, (2) measurement of the pleural pressure at high temporal resolution. The prior art measures the severity of cardiac decompensation and the severity of the respiratory effort as the mean peak-to-peak swing of the respiratory signal, over few breath cycles, i.e., over more than 10 sec. Unlike the prior art, the present invention provides treatment with precise synchronization of cardiac pacing to the respiratory dynamic, with a significantly better resolution of less than 30 milliseconds. The present invention uses a different technique than the prior art, the technique being based on simultaneous measurement of both the intravascular pressure and the intravascular volume. The instantaneous pleural pressure is calculated from these two measurements, after the elastic vessel compliance was calibrated.

In contrast to the prior art, the synchronization of cardiac pacing to the respiratory dynamics is not aligned to a simple segmentation of breathing cycle to inspiration and expiration phase, but rather uses more precise assessment of the pleural pressure dynamics and defines a threshold that relates to the pleural pressure level, as depicted in FIG. 3. Inspiration is defined as the time interval of inhalation, when the intrathoracic pressure drops from a pressure close to zero to the lowest negative intrathoracic pressure. Similarly, pacing during the early expiratory phase is ineffective, since the intrathoracic pressure is very low at the beginning of the expiration phase. The appropriate pacing widow crosses the inspiration and expiration phases, and starts before the end expiration and ends after the beginning of inspiration. This segmentation of the respiratory cycle is unique to this embodiment, in comparison the all the other suggested pacing of the heart (such as U.S. Pat. Nos. 8,509,902, 8,483,833).

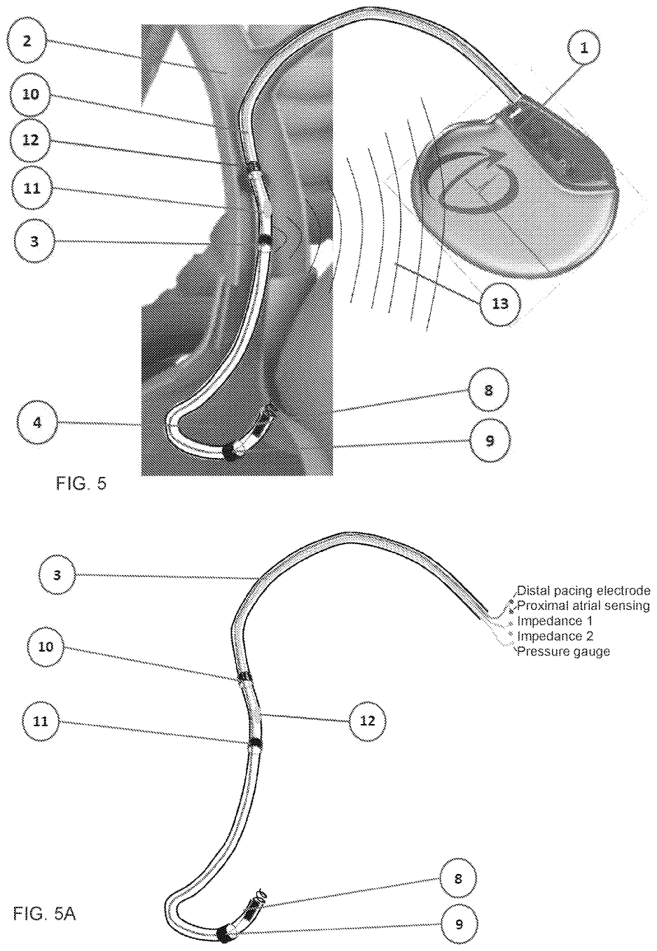

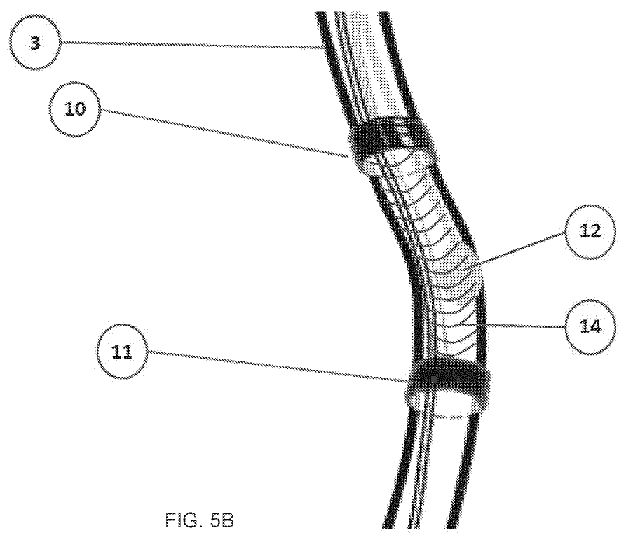

The catheter includes a combination of sensors that assist in the diagnosis and quantification of heart failure severity and define when to pace the heart within the respiratory cycle. This includes:
1. The intracardiac ECG and the respiratory induced changes in the intracardiac electrocardiography (8 and 9 in FIG. 5).
   The ECG is essential for the precise triggering of cardiac pacing, in the appropriate time after the last cardiac QRS signal.
   Monitoring the changes in the heart QRS axis during the respiratory cycle and in the sinus node rate and rhythm.
2. Intravascular pressure measurements, within any of the intrathoracic arterial or venous vessels (12 in FIG. 5), since the pressures within the intrathoracic vessels increases with cardiac decompensation, and the intravascular pressure is modulated by the changes in the intrathoracic/pleural pressure. The heart and the great vessels are within the mediastinum and are surrounded by the intrathoracic pressure. Thus, breathing changes all the intrathoracic, intravascular, and intracardiac pressures. In addition to the effect of the respiratory effort on the peak-to-peak swing of the intravascular pressure with the respiration, the respiratory effort also affects the mean intravascular pressure. An increase in the respiratory effort is associated with an increase in the mean intravascular pressure, since it increases the pulmonary vascular resistance.
3. Intravascular volume measurement, within any of the intrathoracic arterial or venous vessels, utilizing the impedance technology (10, 11 in FIG. 5). Cardiac decompensation is associated with an increase in the intravascular pressure and volume. However, there is no simple linear relationship between the two, but an exponential one. At low intravascular pressure, the vessel compliance is large, and significant changes in the volume are associated with smaller changes in the intravascular pressure. Thus, the intravascular volume is more sensitive to the severity of congestion.
4. Assessment of pulmonary congestion by the impedance technology, between any electrodes along the catheter and the device (13 in FIG. 5).
5. Measurement of the intrathoracic (pleural) pressure. The peak-to-peak amplitude of the pleural pressure is used as a surrogate to the respiratory effort. Moreover, the pleural pressure defines when to pace the heart, based on the instantaneous absolute value of the pleural pressure (and not just the peak-to-peak amplitude).
   The pleural pressure (PPL) is assessed from the measurements of the intravascular volume (VV) and pressure (PV):

$${P}_{PL} ( t ) = {P}_{V} ( t ) - \frac{{V}_{V}}{{C}_{V}} \qquad 15$$

where CV is the vessel compliance, and it is constant.

The vessel compliance (CV) can be estimated noninvasively, by asking the patient to stop breathing at least two times, and to generate different mouth pressure each time. From two simultaneous measurements of intravascular volume (VV1,VV2), intravascular pressure (PV1,PV2) and the pressure at the mount (PM1,PM2) it is 20 possible to calculate the vessel compliance:

$${C}_{V} = \frac{{V}_{V 1} {P}_{M 2} - {V}_{V 2} {P}_{M 1}}{{P}_{V 1} {P}_{M 2} - {P}_{V 2} {P}_{M 1}}$$

However, more calibration data points will improve the precision of the calibration and will enable to consider nonlinear compliance (a decrease in the vessel compliance at large intravascular volumes).

Measurement of the pleural pressure is based on the fact that the compliances of the central blood vessels (as the vena-cava, pulmonary artery and aorta) are determined by the passive elastic properties and are practically constants within a period of several months.

$${C}_{V} = \frac{{V}_{V} ( t )}{{P}_{V} ( t ) - {P}_{PL} ( t )} \qquad ( 1 )$$

where C_{V} is the vessel compliance, V_{V}(t) is the vessel blood volume, P_{V}(t) is the intravascular pressure and P_{PL}(t) is the pleural pressure. Knowing the intravascular volume (V_{V}(t)) and pressure (P_{V}(t)) enable to calculate the pleural pressure:

$${P}_{PL} ( t ) = {P}_{V} ( t ) - \frac{{V}_{V}}{{C}_{V}} \qquad ( 2 )$$

The vessel compliance can be assessed noninvasively, by asking the patient to stop breathing at least at two pressures. When there is no air-flow, the alveolar pressure (PA¬) is equal to the pressure measured at the mouth (PM). Under this condition also the pleural pressure is fixed and is equal to:

$${P}_{PL} = {P}_{M} \frac{{C}_{W}}{{C}_{W} + {C}_{L}} = {C}_{LW} {P}_{M} \qquad ( 3 )$$

where C_{L} and C_{W} are the lung and chest wall compliance, respectively, and the coefficient C_{LW} is constant (C_{LW}=C_{W}/(C_{W}+C_{L}).

Substituting Eq. 3 in Eq. 1 and rearranging yields the equation for the blood vessel volume (V_{V}):

V_{V}(t)=C_{V}(P_{V}(t)−C_{LW}P_{M}) (4)

Equation 4 includes two constants: C_{V} and C_{LW}. Thus, from 2 simultaneous measurements of intravascular volume (V_{V1}, V_{V2}), intravascular pressure (P_{V1},P_{V2}) and the pressure at the mount (P_{M1},P_{M2}) it is possible to calculate the vessel compliance:

$${C}_{V} = \frac{{V}_{V 1} {P}_{M 2} - {V}_{V 2} {P}_{M 1}}{{P}_{V 1} {P}_{M 2} - {P}_{V 2} {P}_{M 1}} \qquad ( 5 )$$

These measurements (V_{V1}, V_{V2}, P_{V1}, P_{V2}, P_{M1}, P_{M2}) can be the mean measurements over one or several heart cycles.

The large elastic vessels compliance (C_{V}) changes slowly with age over the years, and therefore there will be a need for calibration every several months. It will be part of the regular practice of the pacemaker follow-up, to recalibrate the coefficient of the vessel compliance, in addition to checking the pacing threshold.

### Additional Remarks

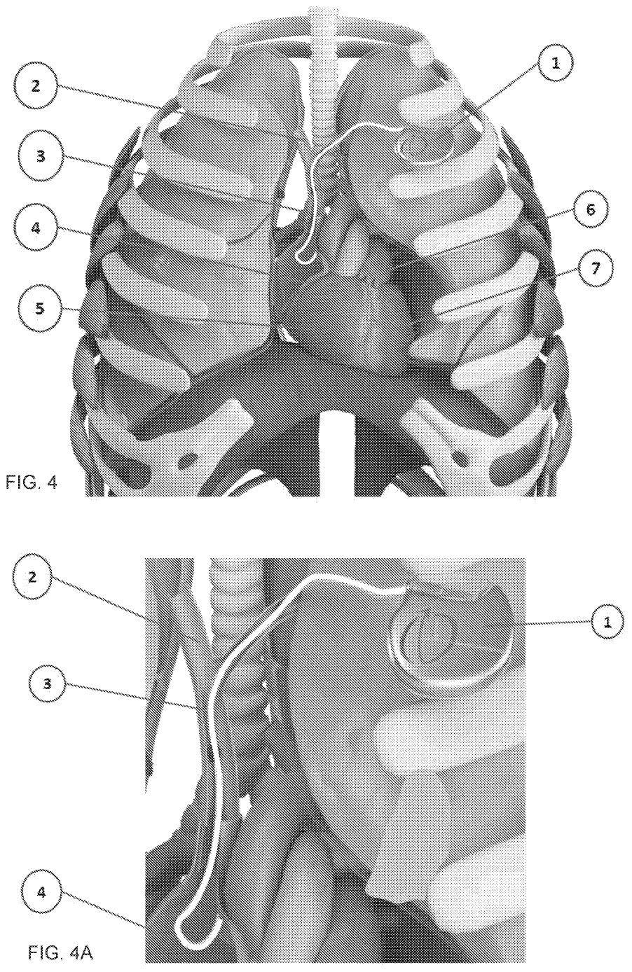

The catheter can be implanted in each of the heart chambers (4, 5, 6, or 7 in FIG. 4). The preferred site is the right atrium (4) if the patient has normal sinus rhythm. If the patient has atrial fibrillation the preferred site is the right ventricle (5 in FIG. 4).
Several catheters can be used. Some can provide the sensing and others the pacing. Sensing can be made simultaneously in the vena cava and within the heart chamber (as the right atrium and right ventricle). Pacing can be done in both the atrium and the ventricle in case of atrioventricular block.
The intravascular volume, and intravascular pressure, pulmonary impedance and changes in the cardiac rhythm can be used in combination or separately for the analysis of cardiac decompensation severity.
The pleural pressure, intravascular volume, intravascular pressure, pulmonary impedance and changes in the cardiac rhythm can be used in combination or separately to define the inspiration and expiration phases, and may assist in determining the pacing time period within the respiratory cycle.

Applications of the invention include, without limitation, treatment of heart failure patients including all heart failure types, treatment of low cardiac output and lung congestion. The invention is useful for:
Early diagnosis of decompensation, for prevention of deterioration. It can monitor the early signs of deterioration, as increase in intravascular volume, increase in the respiratory effort (pleural pressure).
Adaptive (real time) modulation of cardiac pacing based on the assessment of the respiratory effort severity and cardiac decompensation severity.
Real-time diagnosis, bases on invasive respiratory and hemodynamic indices, for remote medicine.
Precise definition the pacing period within the respiratory cycle, based on the pleural pressure
Simplification of the implantation, by insertion of a single catheter of both sensing and pacing, and reducing the short and long-term complications of implanted catheters, by utilizing a single catheter.

1-19. (canceled)

20. A system for treatment of cardiac problems, comprising:
at least one intrathoracic sensor operative to sense a parameter related to respiratory dynamics;
a catheter; and
a control unit in communication with said intrathoracic sensor, said control unit configured to modulate a cardiac rhythm of a patient in response to said sensed parameter,
wherein said at least one intrathoracic sensor is located along said catheter.

21. The system according to claim 20, wherein said modulating is configured to decrease the relative probability of cardiac contraction when the surrounding pleural pressure is more negative and shift cardiac contraction probability to times with a higher surrounding pleural pressure.

22. The system according to claim 21, wherein said modulating is without being aligned to a simple segmentation of breathing cycle to inspiration and expiration phases.

23. The system according to claim 20, wherein said intrathoracic sensor comprises an intravascular sensor.

24. The system according to claim 23, wherein said at least one intravascular sensor is configured to assess pleural pressure and/or changes thereof from inside a blood vessel.

25. The system according to claim 24, configured to assess said pleural pressure and/or changes thereof based on simultaneous measurement of intravascular pressure and the intravascular volume.

26. The system according to claim 24, wherein said pleural pressure is calculated based on at least two of simultaneous measurements of said intravascular pressure, intravascular blood volume and mouth pressure.

27. The system according to claim 20, wherein said respiratory dynamics comprise pleural pressure dynamics and wherein said control unit is configured to use said pleural pressure dynamics to define a threshold that relates to a pleural pressure level.

28. The system according to claim 27, wherein said threshold is used to define a pacing window which starts before an end expiration and ends after the beginning of inspiration.

29. The system according to claim 20, comprising a device for cardiac pacing in communication with said controller, said device for cardiac pacing comprising at least one pacing electrode and wherein said pacing electrode is mounted on and/or in said catheter.

30. The system according to claim 29, wherein said at least one intrathoracic sensor is configured to measure a change in lung congestion by measurement of impedance between said at least one electrode along the catheter and electrodes on a surface of said device for cardiac pacing.

31. The system according to claim 29, wherein said at least one pacing electrode is positioned at a distal portion of said catheter and said at least one intrathoracic sensor is positioned proximal to said at least one pacing electrode.

32. The system according to claim 29, wherein said at least one intrathoracic sensor senses intrathoracic pressure, with higher pressure values being closer to zero than lower pressure values, and wherein pacing of said device for pacing is controlled by said control unit to increase a number of heart beats when the intrathoracic pressure is close to zero relative to the number of heart beats during more negative intrathoracic pressure.

33. The system according to claim 29, wherein pacing of said device for pacing is controlled by said control unit (a) to remove fluids from a lung of the patient and/or (b) to reduce pressures within pulmonary vessels of the patient in an amount sufficient to reduce a respiratory effort and/or dyspnea.

34. The system according to claim 20, wherein modulation of the cardiac rhythm depends on one or both of (a) severity of a respiratory effort and (b) a lung congestion of the patient.

35. The system according to claim 34, wherein said intrathoracic sensor comprises a pressure sensor within an intrathoracic vessel or a heart chamber, that records and measures respiratory waves, and wherein severity of the respiratory effort is defined as peak-to-peak amplitude of the respiratory waves.

36. The system according to claim 20, wherein said at least one intrathoracic sensor is positioned at least 8-10 cm from a distal end of said catheter.

37. The system according to claim 20, wherein said modulating is controlled by said control unit to reduce resistance to blood flow within pulmonary circulation of the patient and/or reduce a respiratory effort in an amount sufficient to alleviate right and/or left ventricle workloads.

38. The system according to claim 20, further comprising an electrocardiogram (ECG) sensor, and wherein said control unit comprises a long-term central control system configured to determine a respiratory interval of elicited pacing, based on past history of ECG recordings, heart rate and/or arrhythmias.

39. The system according to claim 20, wherein said at least one intrathoracic sensor is configured to measure a value and/or a change in a diameter or volume of a blood vessel or a cardiac chamber.

40. The system according to claim 39, wherein said at least one intrathoracic sensor configured for sensing measuring a value and/or a change in diameter or volume measures an electrical impedance between at least two sensing electrodes, and wherein an increase in the diameter of the blood vessel decreases the electrical impedance.

41. The system according to claim 40, wherein said change is used as an index for severity of blood congestion and wherein the impedance is a hyperbolic function of the volume.

42. The system according to claim 39, wherein said at least one intrathoracic sensor comprises four electrodes wherein two of said electrodes are used for injecting alternating currents or voltage, and two other electrodes are used for sensing voltage or current.

43. The system according to claim 39, wherein said at least one intrathoracic sensor comprises two electrodes used to impose alternating voltages or currents, and to measure the current or voltage, the measured value of current or voltage being dependent on the volume within the blood vessel.

44. The system according to claim 20, wherein said control unit is operative to determine at least one index of cardiac decompensation, of severity of a respiratory effort and/or of lung congestion.

45. The system according to claim 44, wherein said at least one index is determined based on determination of the intrathoracic pressure from the simultaneous measurements of values and/or changes in the intravascular pressure and/or intravascular blood volume.

46. The system according to claim 20, wherein said control unit uses an estimate of a pleural pressure from measurements of said intrathoracic sensor using a previously provided vessel compliance value.

47. The system according to claim 46, wherein said compliance value is calculated based on at least two of simultaneous measurements of intravascular pressure, intravascular blood volume and mouth pressure.

48. The system according to claim 20, wherein said control unit is configured to:
(a) determine a value and/or a change in pleural pressure at a temporal resolution of better than 30 milliseconds; and
(b) modulate a cardiac rhythm at various pleural pressures in response to said detected change and/or value.

49. The system according to claim 20, further comprising an electrocardiogram (ECG) sensor, and wherein said control unit uses cardiac electrical activity sensed by said ECG sensor for appropriate timing of pacing in cadence with the patient's autonomous cardiac pacing, and/or for precise triggering after the last QRS signal.

50. An apparatus for treatment of cardiac problems, comprising:
an intravascular sensor; and
a control unit coupled to said intravascular sensor and configured to:
(a) determine a value and/or a change in pleural pressure at a temporal resolution of better than 30 milliseconds; and
(b) differently modulate a cardiac rhythm at various pleural pressures in response to said detected value and/or change.
